(19) United States
(12) Patent Application Publication
(10) Pub. No.: US 2023/0065369 A1
(43) Pub. Date: Mar. 2, 2023

(54) SYSTEM AND METHOD FOR HEALTHCARE PRODUCT SECURE SERIALIZATION AND PRODUCT VALIDATION

(71) Applicant: Advasur, LLC, Rockville, MD (US)

(72) Inventor: John Randall Hoggle, Rockville, MD (US)

(21) Appl. No.: 17/900,217

(22) Filed: Aug. 31, 2022

Related U.S. Application Data
(60) Provisional application No. 63/238,866, filed on Aug. 31, 2021.

(51) Int. Cl. G06Q 20/40; G06F 21/57

(52) U.S. Cl. CPC G06Q 20/401; G06F 21/577; G06F 2221/034

(57) ABSTRACT

Systems and methods for raw material serialization and validation of medical products and raw materials are provided herein. These systems allow participants in the supply chain to add relevant information to a private ledger that can accessed in order to validate products and determine vulnerabilities in the supply chain such as the presence of counterfeit products and raw materials.

### BACKGROUND OF THE INVENTION

All Healthcare products currently produced and marketed can be counterfeited, and drug products may be diverted, subpotent, substandard, adulterated, misbranded, or expired. Despite herculean efforts of money and expertise by countries and the private sector to protect authentic products, it is estimated that over 10% of the worldwide pharmaceutical supply contains counterfeit product. Studies continue to demonstrate even with the billions of dollars spent to protect the pharmaceutical supply chain, the impact of counterfeit healthcare products on the global economy and the risk such counterfeited and diverted have on the buyer/consumer has not been mitigated.

The products most at risk for counterfeit are the higher priced and higher volume healthcare products. Prescription Pharmaceutical Products are impacted more than other healthcare products, where globally the counterfeit rate still exceeds twelve percent (12%) and the US is still estimated to have three percent (3%) or more counterfeit product within its supply chain.

Pharmaceutical product supply chain regulatory compliance is becoming a focus for pharmaceutical manufacturers and supply chain partners, wholesalers, and dispensers across the world for finished products for commercialization. Most developed and developing economies have either already laid out the regulatory road map for serialization or are in the process of doing so.

The regulatory push to secure the pharmaceutical supply chain comes as a result of rising drug-related criminal activities and supply chain inefficiencies. Efforts are aimed at addressing drug counterfeits and unauthorized parallel supply chains, improving supply chain visibility, difficulty in tracking returns or recalls, and the paucity of data-driven tools for predicting patient behavior.

The US is leading the way with the Drug Supply Chain Security Act (DSCSA) passed in 2013. The DSCSA road map for end-to-end traceability is stretched across a period of 10 years, with deliverables outlined for all entities of the supply chain for finished product.

There is no reference within the law for raw materials validation, authentication, certification, or serialization. In the US, lot-level traceability of finished product began in January 2015 under the act. Package level serialization for finished product shipped by pharmaceutical manufacturers was required in the law and became effective November 2018. In November 2019, the requirement for pharmaceutical wholesalers to be able to receive serialized manufacturers product data became effective. Dispensers are required in November 2020 to be able to receive serialized manufacturers product data directly from manufacturers or through Authorized Distributor of Record (ADR) wholesalers. The entire supply chain is required to electronically integrate the serialized product data into their internal operations and be able to upon regulatory request generate reports demonstrating comprehensive mastery of the serialize product data and information by November 2023.

In parallel, the European Union (EU) has followed suit with a compliance requirement by enacting the Falsified Medicines Directive (FMD). Unit-level serialization and dispenser authentication have been mandated, with an initial deadline of February 2019, but it has not yet been implemented. By 2012, India's Directorate General of Foreign Trade (DGFT) also mandated serialization of secondary and tertiary levels and set guidelines for the reporting of export shipments. Several other countries have drafted similar regulations for manufacturing and imports.

With India being a major exporter to the US, Europe, and other regulated markets, these regulations have a significant impact on Indian pharmaceutical manufacturers. Implementing serialization across the global supply chain requires a holistic approach because of the large number of differing requirements for all participants within the market. The scale of packaging operations, artwork-level changes, integrated information flow, and availability of the right internal and external resources add to the complexity of reducing counterfeit products.

At least twenty-seven nations have created different serialization system requirements for manufacturers commercializing products in their country. The pharmaceutical manufacturers, distributors, and dispensers trading partners in each country must develop detailed project plans and well-defined programs to ensure accountability and meet delivery timelines. Each Trading Partner in each country must set-up cross-functional teams of packaging, engineering, IT, quality assurance, and regulatory affairs for seamless implementation. On the ground, implementation starts with upgrading the packaging line with additional equipment, modification of the risk evaluation and mitigation strategies, and adaptation to reduce other inherent risks. Significant time, capital, and thorough planning is required for end-to-end implementation of a serialization and traceability program. Pharmaceutical manufacturers will need to involve both internal and external stakeholders for successful execution.

The Secretary of Health and Human Services (HSS) is responsible for developing standard numerical identifiers (SNIs) for prescription drug packages. The SNIs are applied to a prescription drug at the point of manufacturing and repackaging at the package- or pallet-level, sufficient to facilitate the identification, validation, authentication, and tracking and tracing of the prescription drug. An SNI applied at the point of repackaging is to be linked to the SNI applied at the point of manufacturing and, to the extent practicable, the SNI should be harmonized with international consensus standards for such an identifier.

Various SNIs have been developed for use in the pharmaceutical industry. In the United States most prescription drug packages utilize a serialized National Drug Code (sNDC). Some prescription drugs, such as blood and blood components and certain minimally manipulated human cells, tissues, and cellular and tissue-based products (HCT/Ps), do not currently use NDC numbers. These products use other recognized standards for identification and labeling, such as ISBT 128, which creates a unique identification number for each product package. The SNI may also include expiration dates and/or lot or batch numbers in accordance with GS1 standards for use of Global Trade Item Numbers (GTIN). GTIN is used worldwide by twenty-three industry sectors, including healthcare, and has been adopted by sixty-five countries to uniquely identify pharmaceutical products.

There exists a need to develop systems linking SNIs among entities at different stages of the supply chain, and between product containers at different stages of the supply chain, such as case- and pallet-levels and the package-level sold to consumers.

### SUMMARY OF THE INVENTION

In one aspect, the invention involves a healthcare product serialization system configured to establish supply chain integrity wherein the system allows the generation of an unlimited number of evolving serialization codes for raw materials and products in the supply chain of a final-product, and provides a complete audit record allowing traceability of products in the supply chain. The system comprises comprising a memory storing computer executable modules; a relational database to provide access to data used by the modules; and a processor configured to execute the computer executable modules. The system may include several modules, including a finished-product serialization module, a credentialed party origination module, a private transaction ledger module, a credentialed party authentication module, and a vulnerability assessment module. Also included are methods for product serialization using these systems.

The finished product serialization module is configured to assign a unique finished product serialization code to each finished product in the finished product supply chain. The credentialed party origination module is configured to create accounts of credentialed parties in the supply chain, including at least one or more final-product manufacturer, and optionally including one or more raw material distributors, final-product distributors, final-product re-packagers, and/or end-user dispensers. The private transaction ledger module is configured to securely store a complete transaction history for each finished-product in the supply chain on a private transaction ledger. The credentialed party authentication module configured to provide one or more credentialed parties with controlled access to the private transaction ledger for reading and/or recording transactions to the private transaction ledger. The vulnerability assessment module configured to detect vulnerabilities based on data provided by the finished product serialization model.

### BRIEF DESCRIPTION OF THE DRAWINGS

FIG. 1 shows an example of a serialized National Drug Code (sNDC).

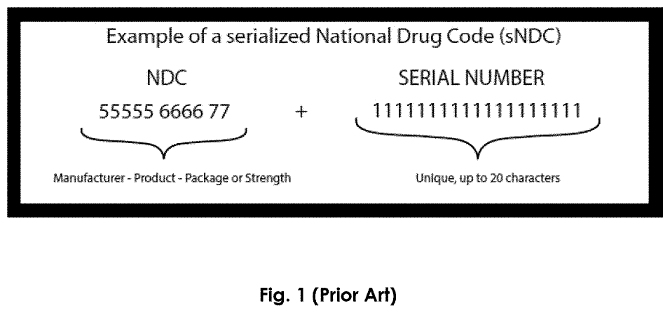

FIG. 2 shows the Automatic Shipping Notification (ANS) documentation flow for finished products under the Drug Quality and Security Act (DQSA).

FIG. 3 shows a data flow diagram for healthcare raw materials and finished products that can be achieved according to an aspect of the invention.

FIG. 4 shows an example of a raw material vulnerability assessment tool according to an embodiment of the invention.

FIG. 5 shows an example of a serialization coding non-deterministic random number generators (NDRNGs) that can be used with the systems and methods described herein, in accordance with an aspect of the invention.

### DETAILED DESCRIPTION OF THE INVENTION

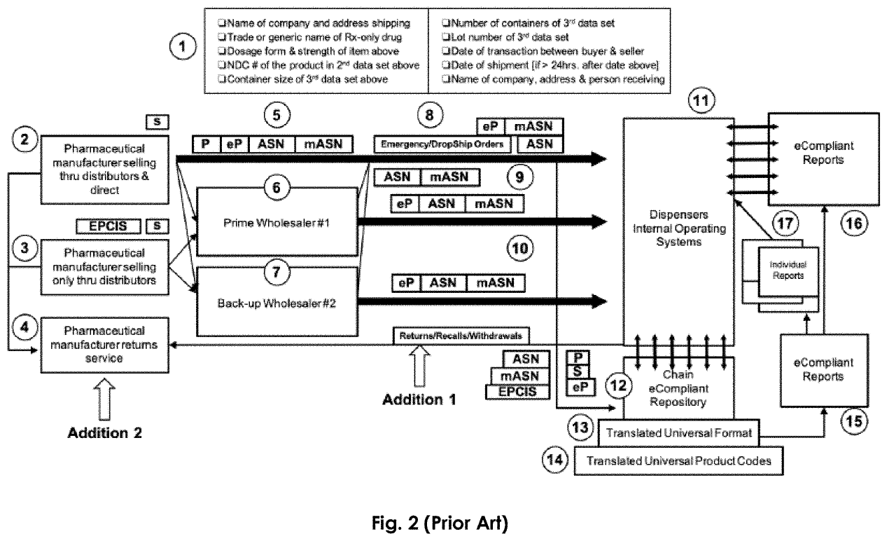

The present invention systems and methods for medical raw material and finished product serialization and validation. Existing systems for documenting the supply chain rely on automatic shipping notifications using a process flow similar to that shown in FIG. 2. The present inventors have identified seventeen DSCSA compliance finished product vulnerability points which are also shown in FIG. 2. These vulnerabilities include data points required for pedigree validation 1, pharmaceutical manufacturers selling through ADRs & Direct 2, pharmaceutical manufacturers selling only through ADRs 3, pharmaceutical manufacturers receiving returned product 4, variety of manufacturer electronic pedigree shipment notification 5, prime vendor wholesaler shipping notifications 6, alternative wholesaler shipping notifications 7, emergency/drop ship orders 8, variety of prime vendor electronic pedigree shipment notification 9, alternative wholesaler electronic pedigree shipment notification 10, variety of direct selling manufacturers electronic pedigree shipment notification 11, chain eCompliant repository 12, translated universal format 13, translated universal product codes 14, store level eCompliant reports 15, chain eCompliant reports 16, and individual government reports 17.

The present inventors have perceived a need to improve on the systems and methods used for documentation flow in healthcare related products. The improved systems provide are applied to serialization and validation of raw materials and finished products in the supply chain for a finished product, and to serialization and validation in the distribution of products among manufacturers, distributors, and pharmacies.

In one aspect, the invention involves a healthcare product serialization system configured to establish raw-material to finished-product supply chain integrity. The raw materials may include APIs, excipients, additives (e.g., medical additives), residues, and/or other components making up a pharmaceutical or medical product. The initial serialization of the raw materials can take place at the time the raw materials are manufactured, and/or when they are tested by one or more of an API testing lab, excipients testing lab, an additives (e.g., medical additives) testing lab, a residues testing lab, and/or other testing lab.

Raw material serialization establishes a unique code for each serialized raw material. The inclusion of the unique code for each raw material on the private ledger allows validation of the raw material component at any stage of the supply chain. It can also provide instant traceability to and from a source throughout the supply chain. As part of the unique code, or in addition to it, the private ledger may be provided one or more product metrics, such as a quality metric. A quality metric could be information as to the exact date of manufacture, or the time at temperature for a given material. The product metrics may be utilized to validate a product or investigate product faults.

Once raw materials have been serialized, the serialized information is stored on a private ledger and may be accessed or modified by one or more entities provided with access to the system, i.e. credentialed parties. The credentialed parties may have varying levels of access to information within the private ledger. For example, a party in the supply chain for an excipient may be provided with all data related to the excipient but may not be able to access information regarding the API, and vice-versa. However, it would be possible for a downstream distributor to be provided with access to information about both the API and the excipient.

The credentialed parties may include one or more of the following: raw material suppliers, raw material wholesalers, each product design team, each product development team, each manufacturing facility team, each product manufacturing operations team, each product packaging team, each product team, each product health and safety team, each manufacturing warehouse management team, each authorized wholesaler headquarters, each W/S distribution center, each product testing lab, and/or each pharmacy.

A finished product serialization module is provided to assign a unique finished product serialization code to each finished product in the finished product supply chain. Information in the product serial code may be human readable, and other aspects may be machine readable. The finished product serialization code may be used to access information in the private ledger about the product. Because the serialization codes are unique, anyone in the supply chain can easily recognize whether a product has been counterfeited, as the private ledger includes a complete history of the product.

A credentialed party origination module is used to create accounts of credentialed parties in the supply chain, including at least one or more raw-material manufacturer and a final-product manufacturer, and optionally including one or more raw material distributors, final-product distributors, final-product re-packagers, and/or end-user dispensers. A credentialed party authentication module may be used to provide one or more credentialed parties with controlled access to the private transaction ledger for reading and/or recording transactions to the private transaction ledger.

A vulnerability assessment module configured to detect vulnerabilities based on data provided by the raw-material serialization module and the finished product serialization model. The vulnerability assessment module is capable of detecting vulnerabilities or anomalies for products based on data in the private ledger. The module may be configured to detect anomalies based on analysis of the data contained in the private ledger. The anomalies may indicate counterfeiting of drug products and/or raw materials. Additional anomalies may include, but are not limited to, changes in raw material suppliers, changes in manufacturing methods, and changes in inactive ingredients.

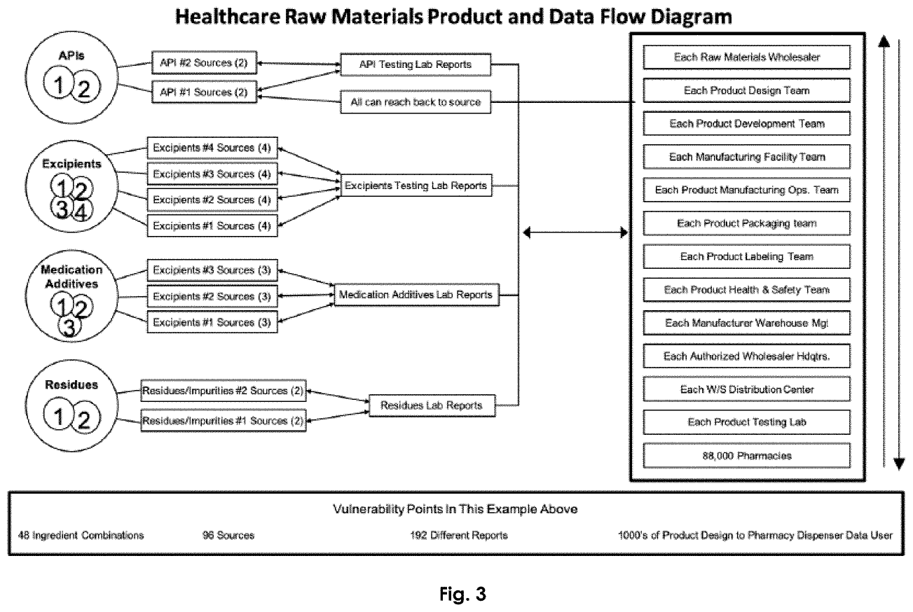

FIG. 3 shows a data flow diagram for healthcare raw materials and finished products that can be achieved according to an aspect of the invention. In this particular case, the drug product includes two active pharmaceutical ingredients (APIs), four excipients, three medical additives. Each of these items is recorded in the private ledger as well as the source for each of these materials. Depending on the material in question, additional information may be included within the private ledger depending on the type of material. For example, the testing lab reports for the API, excipients, additives and or residues/impurities may be entered into the private ledger and associated with their respective materials/sources.

The credentialed parties in this example include each raw material wholesaler, each product design team, each product development team, each manufacturing facility team, each product manufacturing operations team, each product packaging team, each product labelling team, each product health and safety team, each manufacturer warehouse management, each authorized wholesaler headquarters, each W/S distribution center, each product testing lab, and each of the pharmacies (e.g., 88,000 pharmacies). Each of the credentialed parties may have a prescribed level of access. For example, all credentialed parties may be given access to the API and API source information. However, other members may only have been granted access to the testing lab reports for the drug components and could be unable to determine the source of excipients, medical additives, and/or residues.

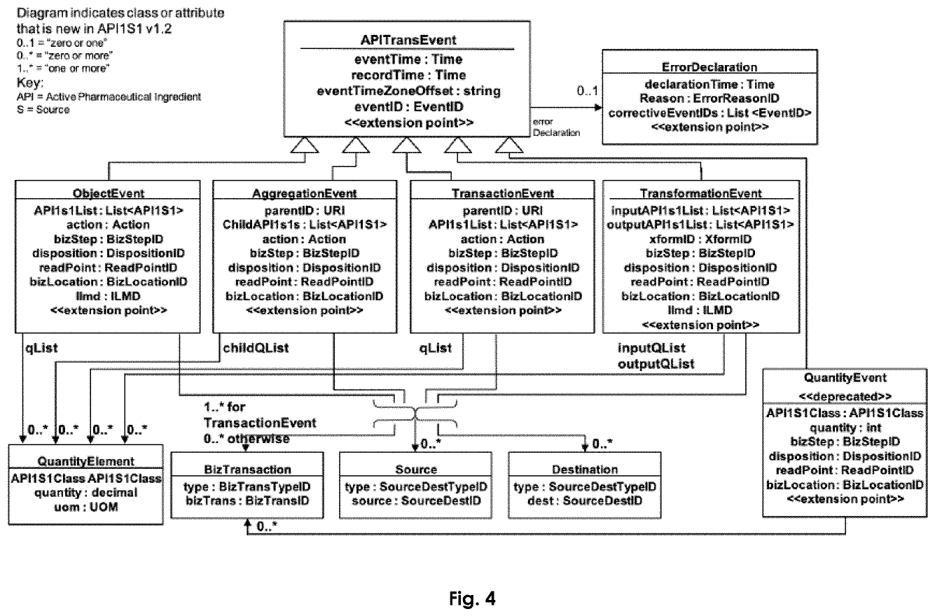

FIG. 4 shows an example of a raw material vulnerability assessment tool according to an embodiment of the invention. In this case, source, destination, and business transactions are monitored. The vulnerability assessment tool shown in FIG. 4 can be used to monitor transactions by monitoring transactions, sources and destinations. The module can track quantity events, object events, aggregation events, transaction events, and transformation events. These events can be associated with a given API and used to track anomalies and vulnerabilities in the supply chain.

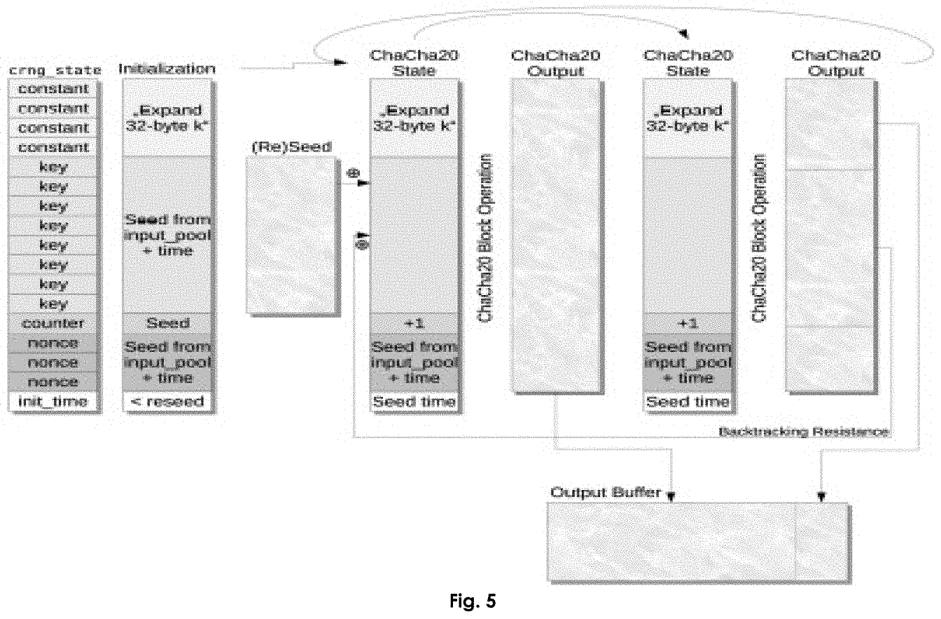

FIG. 5 shows an example of a serialization coding non-deterministic random number generators (NDRNGs). The Serialization Coding Non-Deterministic Random Number Generators (NDRNGs) related to the code that will be seen by the Trading Partner communicators simultaneously due to synchronized unique codes provided to conduct the rules based objective questions with corresponding trading partner response appended to that exact code which serves to provide a historical secure private ledger entry for both trading partners.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A healthcare product serialization system configured to establish supply chain integrity comprising:
a memory storing computer executable modules;
a relational database to provide access to data used by the modules; and
a processor configured to execute the computer executable modules, the computer executable modules comprising:
a finished-product serialization module configured to assign a unique finished product serialization code to each finished product in the finished product supply chain;
a credentialed party origination module for creating accounts of credentialed parties in the supply chain, including at least one or more final-product manufacturer, and optionally including one or more raw material distributors, final-product distributors, final-product re-packagers, and/or end-user dispensers;
a private transaction ledger module configured to securely store a complete transaction history for each finished-product in the supply chain on a private transaction ledger;
a credentialed party authentication module configured to provide one or more credentialed parties with controlled access to the private transaction ledger for reading and/or recording transactions to the private transaction ledger; and
a vulnerability assessment module configured to detect vulnerabilities based on data provided by the finished product serialization model;
wherein the system allows the generation of an unlimited number of evolving serialization codes for raw materials and products in the supply chain of a final-product, and provides a complete audit record allowing traceability of products in the supply chain.

2. The system of claim 1, wherein the system further comprises:
a final-product re-packaging serialization module configured to provide unique serialization codes to re-packaged products within the supply chain.

3. The system of claim 1, wherein the private transaction ledger is implemented on a blockchain.

4. The system of claim 1, wherein a credentialed raw-material manufacturer is provided access to the private transaction ledger that is limited to transactions involving the raw-material, and is prevented from gaining access to information about other raw materials used in the final-product.

5. The system of claim 1, wherein a credentialed final-product manufacturer is provide access to the private transaction ledger for all transactions.

6. The system of claim 1, wherein a credentialed final-product manufacturer is provide access to the private transaction ledger for all transactions.

7. The system of claim 1, wherein the serialization code are provided with packaging of the raw-materials or finished-products.

8. The system of claim 1, wherein the final product formulation matches government approved criteria, the final product is assigned a serialization code that ensures product integrity.

9. The system of claim 1, wherein the serialization code is provided along with a serialized national drug code (sNDC).

10. The system of claim 1, wherein the unique serialization code for a trading partner is generated when ownership is transferred.

11. The system of claim 1, wherein the ownership is transferred via an electronic Pedigree (ePed), Automatic Shipping Notification (ASN), or Electronic Product Code Information System (EPCIS).

12. The system of claim 11, wherein a purchaser receives the unique serialization code along with the electronic Pedigree (ePed), Automatic Shipping Notification (ASN), or Electronic Product Code Information System (EPCIS).

13. A method of healthcare product serialization for establishing raw-material to finished-product supply chain integrity, comprising at least the following steps:
assigning a unique finished product serialization code to each finished product in the finished product supply chain;
creating accounts of credentialed parties in the supply chain, including at least one or more final-product manufacturer, and optionally including one or more raw material distributors, final-product distributors, final-product re-packagers, and/or end-user dispensers;
securely storing a complete transaction history for each finished-product in the supply chain on a private transaction ledger;
providing one or more credentialed parties with controlled access to the private transaction ledger for reading and/or recording transactions to the private transaction ledger; and
detecting vulnerabilities based on data provided by the finished product serialization model,
wherein an unlimited number of evolving serialization codes can be generated for raw materials and products in the supply chain of a final-product, and a complete audit record allowing is provided allowing for traceability from raw-materials to final-products in the supply chain for a final-product.

14. The method of claim 13, further comprising:
providing unique serialization codes to re-packaged products within the supply chain.

15. The method of claim 13, wherein the private transaction ledger is implemented on a blockchain.

16. The method of claim 13, wherein a credentialed raw-material manufacturer is provided access to the private transaction ledger that is limited to transactions involving the raw-material, and is prevented from gaining access to information about other raw materials used in the final-product.

17. The method of claim 13, wherein a credentialed final-product manufacturer is provide access to the private transaction ledger for all transactions.

18. The method of claim 13, wherein a credentialed final-product manufacturer is provide access to the private transaction ledger for all transactions.

19. The method of claim 13, wherein the serialization code are provided with packaging of the raw-materials or finished-products.

20. The method of claim 13, wherein the final product formulation matches government approved criteria, the final product is assigned a serialization code that ensures product integrity.

21. The method of claim 13, wherein the serialization code is provided along with a serialized national drug code (sNDC).

22. The method of claim 13, wherein the unique serialization code for a trading partner is generated when ownership is transferred.

23. The method of claim 13, wherein the ownership is transferred via an electronic Pedigree (ePed), Automatic Shipping Notification (ASN), or Electronic Product Code Information System (EPCIS).

24. The method of claim 13, wherein a purchaser receives the unique serialization code along with the electronic Pedigree (ePed), Automatic Shipping Notification (ASN), or Electronic Product Code Information System (EPCIS).
